(19) United States
(12) Patent Application Publication
(10) Pub. No.: US 2021/0300858 A1
(43) Pub. Date: Sep. 30, 2021

(54) METHOD FOR PRODUCING CARBONIC ESTERS

(71) Applicant: BASF SE, Ludwigshafen am Rhein (DE)

(72) Inventors: Volker Hickmann, Ludwigshafen (DE); Wolfgang Siegel, Ludwigshafen (DE); Markus Jegelka, Ludwigshafen (DE); Eva Katharina Hackemann, Ludwigshafen (DE); Stephanie Renz, Ludwigshafen (DE); Manuel Danz, Ludwigshafen (DE); Stephan Maurer, Ludwigshafen (DE)

(21) Appl. No.: 17/266,438

(22) PCT Filed: Aug. 5, 2019
(86) PCT No.: PCT/EP2019/071007

§ 371 Date: Feb. 5, 2021

(30) Foreign Application Priority Data
Aug. 6, 2018 (EP) 18187517.0

(51) Int. Cl. C07C 68/02

(52) U.S. Cl. CPC C07C 68/02; C07C 2601/14

(57) ABSTRACT

The present invention relates to a process for providing carbonic acid esters of formula (I). Further, the invention relates to the intermediates of the formula (IVa).

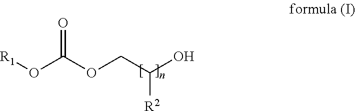

### FIELD OF THE INVENTION

The present invention relates to a process for providing carbonic acid esters of formula (I). Further, the invention relates to the intermediates of the formula (IVa).

### BACKGROUND OF THE INVENTION

The unsymmetrical carbonic acid esters are valuable compounds for the preparation of tooth cleaning agents, mouthwashes, dental rinses, foodstuffs, drinks and cosmetics.

Generally, in the known arts, the carbonic esters can be prepared by reaction of the respective chloroformate and glycol in the presence of pyridine and/or alkali hydroxide.

U.S. Pat. No. 4,509,537 describes the process for providing carbonic esters from phenyl chloroformate and 1,3-propanediol in the solution of pyridine and methylene chloride. In a technical application the often-preferred pyridine results in a number of impairments. Apart from the odor nuisance, when working with pyridine, the purification steps for the separation of pyridine and/or pyridine hydrochloride requires multiple aqueous washes. Thus, the disposal of accumulating pyridine-containing waste water also results in increased expenditure. Additionally, pyridine is labelled as harmful in the material safety datasheet.

The PCT Application No. WO 05/023749 provides the preparation of unsymmetrical carbonic esters by reacting the respective chloroformate and glycol in a homogeneous liquid phase in the presence of an alkali hydroxide. The art describes the addition of chloroformate and an alkali hydroxide to the solvent containing glycol, in the course of 4-5 parallel doses over the period of 2 to 3 hours. The disadvantage associated with the use of an alkali hydroxide is that it requires a lot-wise addition while maintaining the temperature range in order to prevent by-products resulting from hydrolysis of the chloroformate and/or saponification of carbonic ester. Furthermore, N-methyl-pyrrolidone is used as solvent which is a carcinogenic, mutagenic or toxic substance.

It is known that the chloroformates of formula (II) can be obtained from the corresponding alcohol and phosgene; however, certain amounts of impurities are produced by this reaction, especially through chlorination of the respective alcohols, and these impurities must be removed by methods which may unfavorably affect the general economy of the process wherein such chloroformate is used. Thus, in addition to the disadvantages described above, the presence of such impurities in chloroformates of formula (II) may result in generation of further impurities and/or by-products during reaction of the chloroformates of formula (II) with the glycol of formula (V) and may require extensive purification of the desired product

Consequently, methods for the efficient, environmentally responsible, large-scale preparation of these carbonic acid esters of formula (I) are limited and these methods known in the art are often not suitable for the efficient synthesis of carbonic esters of formula (I).

Accordingly, the object of the presently claimed invention is to provide an improved process for the synthesis of carbonic acid esters of formula (I) which overcomes the problems associated with the methods of the prior art. The invention also relates to the intermediates of said process.

### SUMMARY OF THE INVENTION

In an aspect, the present invention relates to a process for preparing a carbonic acid ester of formula (I) and its stereoisomers,

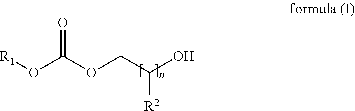

wherein

R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀cycloalkyl and unsubstituted or substituted C₅-C₁₀-cycloalkenyl;

R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

n is 1, 2 or 3;

wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

comprising at least the steps of:
b) reacting a compound of formula (II)

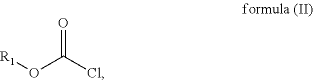

wherein

R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀cycloalkyl and unsubstituted or substituted C₅-C₁₀-cycloalkenyl;

with an imidazole of formula (III),

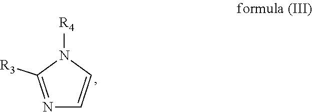

wherein

R₃ is hydrogen or unsubstituted, linear or branched, C₁-C₆-alkyl and R₄ unsubstituted, linear or branched C₁-C₆-alkyl;

to obtain a compound of formula (IV),

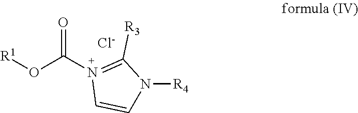

wherein

R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;

R₃ is hydrogen or unsubstituted, linear or branched, C₁-C₆-alkyl; and

R₄ is unsubstituted, linear or branched C₁-C₆-alkyl;

and

b) reacting the compound of formula (I) with a compound of formula (V)

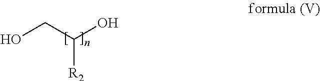

wherein

R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

n is 1, 2 or 3; and

wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

to obtain a compound of formula (I) and its stereoisomers.

In an aspect, the present invention also relates to a compound of formula (IVa) and stereoisomers,

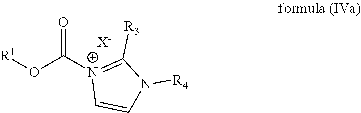

wherein,

when R₃ is unsubstituted, linear or branched, C₁-C₆-alkyl; R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; or

wherein when R₃ is hydrogen; R₁ is selected from the group consisting of unsubstituted or substituted, linear or branched C₄-C₁₀-alkyl or -alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; and

R₄ is unsubstituted, linear or branched, C₁-C₁₀-alkyl;

and X is chloride or bromide.

### Advantageous Effect of the Invention

The present invention provides an advantageous purification protocol to remove chloride impurities by providing an intermediate of formula (IVa).

The method also avoids the use of troublesome solvents like pyridine or N-methyl-pyrrolidone.

### DESCRIPTION OF THE INVENTION

The object is achieved by the processes described in detail hereafter.

In one embodiment, the present invention relates to a process for preparing a carbonic acid ester of formula (I) and its stereoisomers,

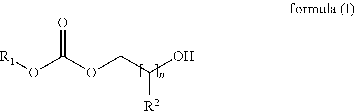

wherein

R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀cycloalkyl and unsubstituted or substituted C₅-C₁₀-cycloalkenyl;

R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl and unsubstituted or substituted C₅-C₁₀-cycloalkyl and unsubstituted or substituted C₅-C₁₀-cycloalkenyl;

n is 1, 2 or 3;

wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

comprising at least the steps of:

a) reacting a compound of formula (II)

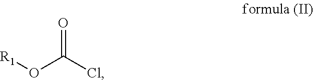

wherein

R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀cycloalkyl and unsubstituted or substituted C₅-C₁₀-cycloalkenyl;

with an imidazole of formula (III),

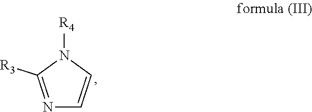

wherein

R₃ is hydrogen or unsubstituted, linear or branched, C₁-C₆-alkyl and R₄ is unsubstituted, linear or branched, C₁-C₆-alkyl;

to obtain a compound of formula (IV),

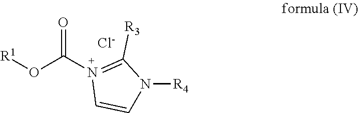

wherein

R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀cycloalkyl and unsubstituted or substituted C₅-C₁₀-cycloalkenyl;

R₃ is hydrogen or unsubstituted, linear or branched, C₁-C₆-alkyl; and

R₄ is unsubstituted, linear or branched, C₁-C₆-alkyl;

and

b) reacting the compound of formula (IV) with a compound of formula (V)

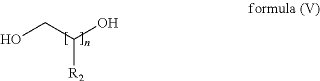

wherein

R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

n is 1, 2 or 3; and

wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

to obtain a compound of formula (I) and its stereoisomers.

Starting materials used in the process are commercially available or can be prepared by methods known in the literature.

The “present invention”, “invention” or “process of the present invention” refers to one or more of the steps (a) and (b).

Although the present invention will be described with respect to particular embodiments, this description is not to be construed in a limiting sense.

Before describing in detail exemplary embodiments of the present invention, definitions important for understanding the present invention are given. As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

Unless otherwise indicated, the following definitions are set forth to illustrate and define the meaning and scope of the various terms used to describe the invention herein and the appended claims. These definitions should not be interpreted in the literal sense as they are not intended to be general definitions and are relevant only for this application.

It will be understood that “substitution”, “substituted” or “substituted with” means that one or more hydrogens of the specified moiety are replaced with a suitable substituent and includes the implicit proviso that such substitutions are in accordance with the permitted valence of the substituted atom and the substituent and results in a stable compound.

When any variable (for instance, R₁, R₂, R₃, R₄, R₅ etc.) or substituent has more than one occurrence, its definition on each occurrence is independent at every other occurrence. Also, combinations of substituents and/or variables are permissible, only if such combinations result in stable compounds.

The term “independently”, when used in the context of selection of substituents for a variable, it means that where more than one substituent is selected from many possible substituents, those substituents may be the same or different.

Salts of the compounds according to the invention can be formed in a customary manner, for example, by reacting the compound with an acid of the anion in question if the compounds according to the invention have a basic functionality or by reacting acidic compounds according to the invention with a suitable base.

The organic moieties or groups mentioned in the above definitions of the variables are like the term halogen—collective terms for individual listings of the individual group members. The term “Cv-Cw” indicates the number of carbon atom possible in each case.

The term “C₁-C₁₀-alkyl” refers to a straight-chained or branched saturated hydrocarbon group having 1 to 10 carbon atoms, for example, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The term “C₃-C₁₀-alkenyl” refers to a straight-chain or branched unsaturated hydrocarbon radical having 2 to 10 carbon atoms and a double bond in any position. Examples are “C₂-C₄-alkenyl” groups, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₃-C₁₀-alkynyl” refers to a straight-chain or branched unsaturated hydrocarbon radical having 2 to 10 carbon atoms and containing at least one triple bond. Examples are “C₂-C₄ alkynyl” groups, such as ethynyl, prop-1-ynyl, prop-2-ynyl, but-1-ynyl, but-2-ynyl, but-3-ynyl, 1-methyl-prop-2-ynyl.

The term “C₅-C₁₀-cycloalkyl” refers to monocyclic saturated hydrocarbon radicals having 5 to 10 carbon ring members, such as cyclopropyl, cyclobutyl, cyclopentyl, cyclohexyl, cycloheptyl or cyclooctyl.

The term “C₅-C₁₀-cycloalkenyl” refers to monocyclic unsaturated hydrocarbon radical having to 10 carbon ring members and a double bond in any position, for example cyclobutenyl, cyclopentenyl, cyclohexenyl or cyclooctenyl.

The term “substituted”, if not specified otherwise, refers to substituted with 1, 2 or maximum possible number of substituents. If substituents are more than one, then they are independently from each other are same or different, if not mentioned other-wise.

Meaning of the terms that are not defined herein are generally known to a person skilled in the art or in the literature.

In another embodiment, the present invention provides a process, wherein R₁ is selected from group consisting of methyl, ethyl, 1-propyl, 1-butyl, 1-pentyl, 1-hexyl, isopropyl, isobutyl, tertiary butyl, isopentyl, 2-methylbutyl and 3-methylbutyl which are each unsubstituted or substituted by 1, 2 or 3 substituents selected from the group consisting of oxo, —F, —NO₂, —CN, —CF₃, —C(═O)CH₃, —C(═O)OCH₃, —NH—C(═O)(CH₃), —CH₂-phenyl, -phenyl; and cyclopropyl, cyclobutyl, cyclopentyl, cyclohexyl, cyclohexenyl and cyclooctyl which are each unsubstituted or substituted by 1, 2, 3, or 4 substituents selected from the group consisting of methyl, ethyl, 1-propyl, 1-butyl, 1-pentyl, 1-hexyl, isopropyl, isopropenyl, isobutyl, tertiary butyl, isopentyl, 2-methylbutyl, 3-methylbutyl, -methoxy, -ethoxy, —F, —NO₂, —CN, —CF₃, —C(═O)CH₃, —C(═O)OCH₃, —NH—C(═O)CH₃.

More preferably, R₁ is cyclohexyl which is substituted by 1 or 2 substituents selected from the group consisting of methyl, ethyl, 1-propyl, isopropyl, isopropenyl and isobutyl.

Most preferably, R₁ is cyclohexyl which is substituted by methyl and isopropyl.

In yet another embodiment, the present invention provides a process, wherein R₂ is hydrogen or selected from group consisting of methyl, ethyl, 1-propyl, 1-butyl, 1-pentyl, 1-hexyl, isopropyl, isobutyl, tertiary butyl, isopentyl, 2-methylbutyl, 3-methylbutyl, cyclopropyl, cyclobutyl, cyclopentyl, cyclohexyl, cyclohexeny and cyclooctyl which are each unsubstituted.

In another embodiment, the present invention provides a process, wherein n is 1, 2 or 3. Preferably, n is 1.

Preferably, when n is 2 or 3, the R₂, independently, is hydrogen or selected from the group consisting of methyl, ethyl, 1-propyl, 1-butyl, 1-pentyl, 1-hexyl, isopropyl, isobutyl, tertiary butyl, isopentyl, 2-methylbutyl, 3-methylbutyl, cyclopropyl, cyclobutyl, cyclopentyl, cyclohexyl and cyclooctyl, which are each unsubstituted.

In yet another embodiment, the present invention provides the process, wherein R₃ is selected from group consisting of hydrogen, methyl, ethyl, 1-propyl, 1-butyl, 1-pentyl, 1-hexyl, isopropyl, isobutyl, tertiary butyl, isopentyl, 2-methylbutyl and 3-methylbutyl. Preferably, R₃ is hydrogen or methyl.

In yet another embodiment, the present invention provides the process, wherein R₄ is selected from group consisting of methyl, ethyl, 1-propyl, 1-butyl, 1-pentyl, 1-hexyl, isopropyl, isobutyl, tertiary butyl, isopentyl, 2-methylbutyl and 3-methylbutyl. In the most preferred embodiment, R₄ is methyl.

In another embodiment, the present invention provides the process, wherein the imidazole of formula (III) is selected from the group consisting of 1-methyl imidazole, 1-ethyl imidazole, 1-propyl imidazole, 1-isopropyl imidazole, 1-butyl imidazole, and 1,2-dimethyl imidazole.

In a preferred embodiment, the present process provides the process, wherein the imidazole of formula (III) is 1,2-dimethyl imidazole or 1-methyl-imidazole.

In yet another embodiment, the present invention provides the process, wherein in step a) the molar ratio of the imidazole of formula (III) to the compound of formula (II) is in the range of ≥0.05:1.0 to ≤3.0:1.0 or preferably in the range of ≥0.06:1.0 to ≤2.75:1.0 or ≥0.075:1.0 to ≤2.5:1.0 or ≥0.25:1.0 to ≤2.5:1.0 or ≥0.5:1.0 to ≤2.5:1.0; more preferably in the range of ≥0.75:1.0 to ≤2.5:1.0 or ≥0.75:1.0 to ≤2.0:1.0 or ≥1.0:1.0 to ≤2.0:1.0.

In yet another embodiment, the present invention provides the process, wherein at least step a) and step b) are carried out simultaneously.

In yet another embodiment, the present invention provides the process, wherein when at least step a) and step b) are carried out simultaneously, then as a base selected from group consisting of triethylamine, tripropylamine, tributylamine and N,N-diisopropyl-ethylamine can be used. In yet another embodiment the molar ratio of the base and the compound of formula (II) is in the range of ≥1.0:1.0 to ≤3.0:1.0, more preferably 2.0:1.0.

In yet another embodiment, the present invention provides the process, wherein in step a) the temperature is in the range of ≥10° C. to ≤80° C.; preferably the temperature is in the range of ≥15° C. to ≤75° C. or ≥15° C. to ≤70° C. or more preferably in the range of 15° C. to ≤65° C. or ≥15° C. to ≤60° C. or even more preferably in the range of ≥15° C. to ≤55° C. or ≥20° C. to ≤60° C. or ≥20° C. to ≤55° C.

In another embodiment, the present invention provides the process, wherein at least one of the step a) and step b) is carried out in the presence of at least one non-polar solvent. The at least one compound of formula (III) and formula (IV) is dissolved or suspended in at least one non-polar solvent. Preferably the at least one non-polar solvent has dielectric constant in the range of ≥1.5 to ≤6.0 or in the range of ≥1.5 to ≤5.0 or even more preferably in the range of ≥1.5 to ≤4.5.

In a preferred embodiment, the at least one non-polar organic solvent is selected from the group consisting of aliphatic hydrocarbons, aromatic hydrocarbons and ethers.

In yet another preferred embodiment, the suitable aliphatic hydrocarbon is selected from the group consisting of pentane, hexane, heptane, cyclohexane and petroleum ether.

Further, in yet another preferred embodiment, a suitable aromatic hydrocarbon is selected from the group consisting of benzene, toluene and xylene.

In yet another preferred embodiment, the suitable ether solvent is selected from the group consisting of diethyl ether, diisopropyl ether, diethylene glycol dimethyl ether and methyl tert-butyl ether.

More preferably, the at least one non-polar solvent is selected from the group consisting of toluene, xylene, cyclohexane, heptane and methyl tert-butyl ether.

In another embodiment, the present invention provides the process, wherein at least in step b) the molar ratio of the compound of formula (II) to the compound of formula (V) is in the range of ≥1.0:2.0 to ≤1.0:20.0.

In yet another embodiment, the present invention provides the process, wherein at least in step b) the temperature is in the range of ≥10° C. to ≤80° C.; preferably the temperature is in the range of ≥15° C. to ≤75° C. or ≥15° C. to ≤70° C. or more preferably in the range of ≥15° C. to ≤65° C. or ≥15° C. to ≤60° C. or even more preferably in the range of ≥15° C. to ≤55° C. or ≥20° C. to ≤60° C. or ≥20° C. to ≤55° C.

In another embodiment, the present invention provides the process, wherein there may be time intervals of seconds, minutes, hours or days between at least step a) and step b).

In yet another embodiment, the present invention relates to process, wherein at least the compound of formula (IV) is isolated from the at least one non-polar solvent.

In one embodiment, the present invention provides a process wherein the compound of general formula (II) is obtained by reacting a compound of formula (II′) with phosgene

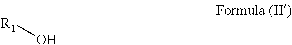

wherein

R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀cycloalkyl and unsubstituted or substituted C₅-C₁₀-cycloalkenyl.

Preferably, R₁ is cyclohexyl or cyclohexenyl which is unsubstituted or substituted by 1, 2 or 3 substituents selected from the group consisting of oxo, methyl, ethyl, 1-propyl, 1-butyl, 1-pentyl, 1-hexyl, isopropyl, isopropenyl, isobutyl, tertiary butyl, isopentyl, 2-methylbutyl and 3-methylbutyl.

When R₁ is cyclohexyl which is substituted by methyl and isopropyl, the at least one compound of formula (IIA)

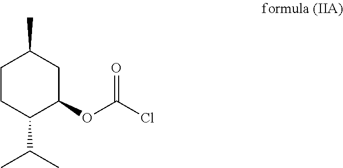

is obtained.

It has been observed that formula (IIA″), menthylchloride, is a potential impurity during the formation of menthylchloroformate (IIA). Also, it has been observed that the amount of formula (IIA″) increases when compound of formula (IIA) is stored for prolonged time or exposed to excessive heat owing to decomposition of compound of formula (IIA).

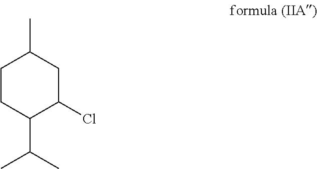

In one embodiment, the present invention provides a process for removing a compound of formula (IIA″) from a compound of formula (IIA) comprising at least the steps of:
A) reacting the mixture comprising compound of formula (IIA) and compound of formula (IIA″) in at least one non-polar solvent

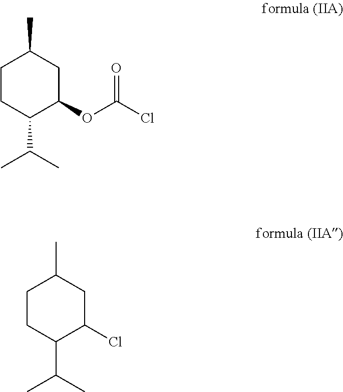

with an imidazole of formula (III)

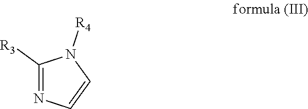

wherein R₃ is hydrogen or unsubstituted, linear or branched, C₁-C₆-alkyl and R₄ is unsubstituted, linear or branched C₁-C₆-alkyl;

to obtain a mixture containing a compound of formula (IVA);

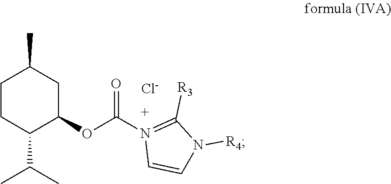

wherein R₃ is hydrogen or unsubstituted, linear or branched, C₁-C₆-alkyl and R₄ is unsubstituted, linear or branched C₁-C₆-alkyl; and
B) optionally, isolating the compound of formula (IVA) from the mixture of step a).

In yet another embodiment, the isolated compound of formula (IVA) can be washed with at least one non-polar solvent. The compound of formula (IVA), so obtained, is free of compound of formula (IIA″). In yet another embodiment, the one non-polar solvent is selected from pentane, hexane, heptane, cyclohexane, petroleum ether, benzene, toluene xylene, diethyl ether, diisopropyl ether, diethylene glycol dimethyl ether and methyl tert-butyl ether.

In another embodiment, the isolated compound of formula (IVA) is reacted with compound of formula (V) in the presence of at least one non-polar solvent and 1-10 mol % imidazole of formula (III).

In yet another embodiment, step A) can be carried out in the presence of compound of formula (V).

In another embodiment, the compound of formula (V) is ethylene glycol or propylene glycol.

In one embodiment, the present invention provides the process for preparing the compound of formula (IA),

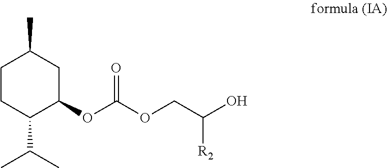

wherein R₂ is hydrogen or methyl;

whereby if R₂ is methyl, the formula (IA) comprises

the compound of formula (Ia)

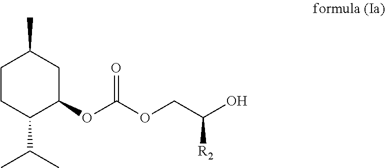

the compound of the formula (Ib)

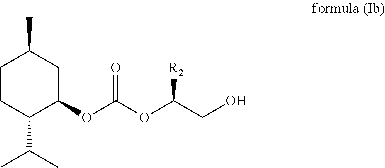

the compound of formula (Ic)

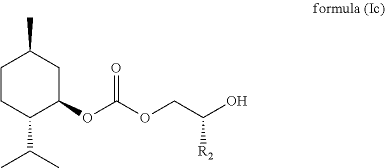

the compound of formula (Id)

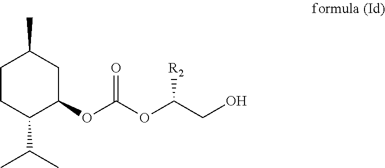

and its stereoisomers,

comprising at least the steps of:

a) reacting the compound of formula (IIA),

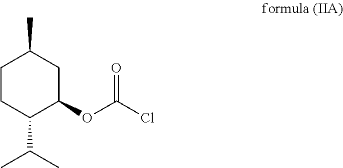

with an imidazole of formula (III) to

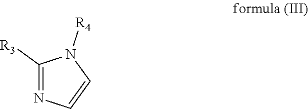

wherein R₃ is hydrogen or unsubstituted, linear or branched, C₁-C₆-alkyl and R₄ is unsubstituted, linear or branched C₁-C₆-alkyl;

to obtain a compound of formula (IVA)

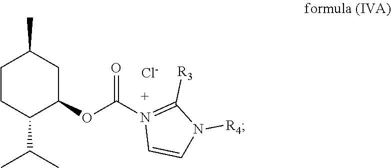

wherein R₃ is hydrogen or unsubstituted, linear or branched, C₁-C₆-alkyl and R₄ is unsubstituted, linear or branched C₁-C₆-alkyl; and

b) reacting the compound of formula (IVA) with the compound of formula (VA) and its stereoisomers;

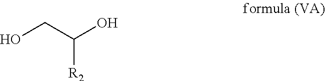

wherein R₂ is hydrogen or methyl;

to obtain the compound of formula (IA) and its stereoisomers.

In yet another embodiment, the present invention provides the process, wherein at least the said compound of formula (I) and formula (IA), respectively, is

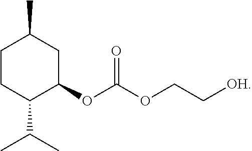 [SMILES: CC(C)[C@@H]1CC[C@@H](C)C[C@H]1OC(=O)OCCO]

In yet another embodiment, the present invention provides the process, wherein at least the said compound of formula (I) and formula (IA) respectively, is

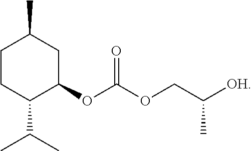 [SMILES: CC(C)[C@@H]1CC[C@@H](C)C[C@H]1OC(=O)OC[C@@H](C)O]

In yet another embodiment, the present invention provides the process, wherein at least the said compound of formula (I) and formula (IA), respectively, is

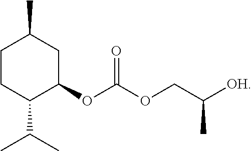 [SMILES: CC(C)[C@@H]1CC[C@@H](C)C[C@H]1OC(=O)OC[C@H](C)O]

In one embodiment, the present invention provides a compound of formula (IVa) and stereoisomers,

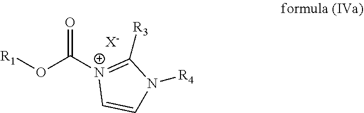

wherein,

when R₃ is unsubstituted, linear or branched, C₁-C₆-alkyl; R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; or

wherein when R₃ is hydrogen; R₁ is selected from the group consisting of unsubstituted or substituted, linear or branched C₄-C₁₀-alkyl or -alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; and

R₄ is unsubstituted, linear or branched C₁-C₁₀-alkyl;

and X is chloride or bromide.

In yet another embodiment, the present invention provides the compound of formula (IVa) and stereoisomers, wherein when R₃ is methyl; R₁ is selected from the group consisting of unsubstituted, linear C₁-C₁₀-alkyl, unsubstituted or substituted C₅-C₁₀-cycloalkyl and unsubstituted or substituted C₅-C₁₀-cycloalkenyl; or wherein when R₃ is hydrogen; R₁ is linear or branched C₄-C₁₀-alkyl or unsubstituted or substituted C₅-C₁₀-cycloalkyl or unsubstituted or substituted C₅-C₁₀-cycloalkenyl.

In yet another embodiment, the present invention provides the compound of formula (IVa) and stereoisomers, wherein when R₃ is methyl; R₁ is selected from the group consisting of unsubstituted, linear C₁-C₁₀-alkyl, unsubstituted or substituted C₅-C₁₀-cycloalkyl and unsubstituted or substituted C₅-C₁₀-cycloalkenyl; or wherein when R₃ is hydrogen; R₁ is linear or branched C₅-C₁₀-alkyl or unsubstituted or substituted C₅-C₁₀-cycloalkyl or unsubstituted or substituted C₅-C₁₀-cycloalkenyl.

In yet another embodiment, the present invention provides the compound of formula (IVa) and stereoisomers, wherein when R₃ is methyl; R₁ is selected from the group consisting of unsubstituted, linear C₁-C₁₀-alkyl, unsubstituted or substituted C₅-C₁₀-cycloalkyl and unsubstituted or substituted C₅-C₁₀-cycloalkenyl; or wherein when R₃ is hydrogen; R₁ is linear or branched unsubstituted C₅-C₁₀-alkyl or unsubstituted or substituted C₅-C₁₀-cycloalkyl or unsubstituted or substituted C₅-C₁₀-cycloalkenyl.

In yet another embodiment, the present invention provides a compound of formula (IVa) and stereoisomers,

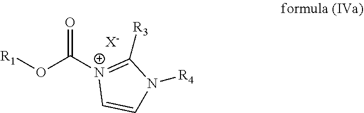

wherein,

when R₃ is unsubstituted, linear or branched, C₁-C₆-alkyl; R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; or

wherein when R₃ is hydrogen; R₁ is selected from the group consisting of unsubstituted or substituted, linear or branched C₄-C₁₀-alkyl or -alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; wherein R₁ is not (CH₃)₂—CH—CH₂— or C₆H₅—CH₂.

and

R₄ is unsubstituted, linear or branched C₁-C₁₀-alkyl;

and X is chloride or bromide.

In yet another embodiment, the present invention provides the compound of formula (IVa) and stereoisomers, wherein the compound of formula (IVa) is

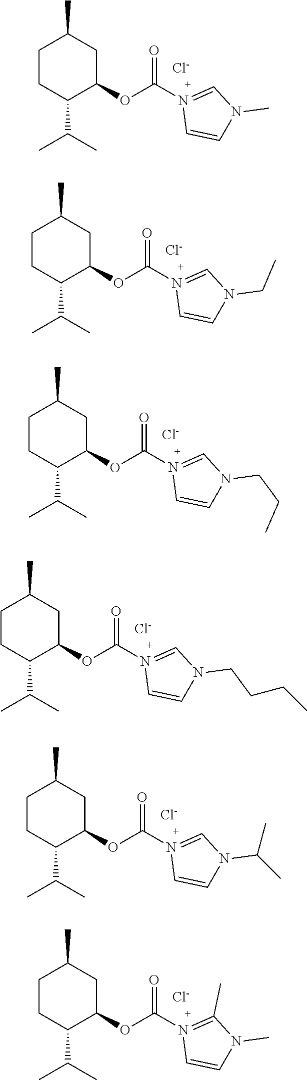

In yet another embodiment, the present invention provides the use of compound of formula (IV) and stereoisomers, wherein the compound of formula (IV),

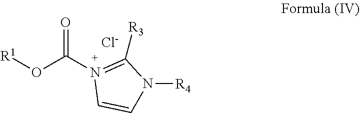

wherein

R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;

R₃ is hydrogen or unsubstituted, linear or branched, C₁-C₆-alkyl; and

R₄ is unsubstituted, linear or branched C₁-C₆-alkyl;

to prepare the compound of formula (I)

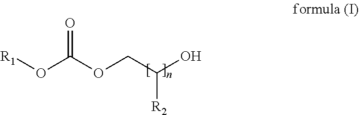

wherein

R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;

R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;

n is 1, 2 or 3;

wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl.

In yet another embodiment, the present invention provides the method of preparing the compound of formula (I) comprising using the compound of formula (IV).

In the following, there is provided a list of embodiments to further illustrate the present disclosure without intending to limit the disclosure to the specific embodiments listed below.
1. A process for preparing a carbonic acid ester of formula (I) and its stereoisomers,

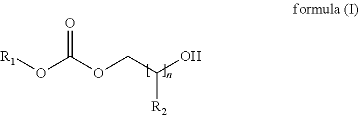

wherein
   R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; R₂ is selected from the group consisting of hydrogen, unsubstituted or substituted, linear or branched C₁-C₁₀-alkyl, unsubstituted or substituted, linear or branched C₂-C₁₀alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;
   n is 1, 2 or 3;
   wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;
   comprising at least the steps of:
      a) reacting a compound of formula (II)

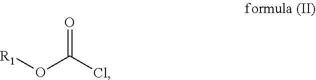

wherein
R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;
with an imidazole of formula (III),

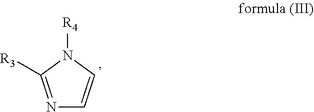

wherein
R₃ is hydrogen or unsubstituted, linear or branched, C₁-C₆-alkyl and R₄ is unsubstituted, linear or branched C₁-C₆-alkyl;
to obtain a compound of formula (IV),

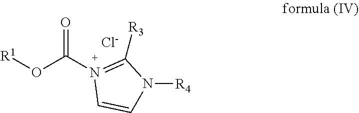

wherein
R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;
R₃ is hydrogen or unsubstituted, linear or branched, C₁-C₆-alkyl; and
R₄ is unsubstituted, linear or branched C₁-C₆-alkyl;
and
b) reacting the compound of formula (IV) with a compound of formula (V)

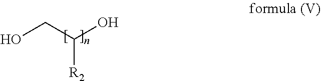

wherein
R₂ is selected from the group consisting of hydrogen, unsubstituted or substituted, linear or branched C₁-C₁₀-alkyl, unsubstituted or substituted, linear or branched C₂-C₁₀alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;
n is 1, 2 or 3; and
wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;
to obtain a compound of formula (I) and its stereoisomers.
2. The process according to embodiment 1, wherein R₁ is selected from group consisting of methyl, ethyl, 1-propyl, 1-butyl, 1-pentyl, 1-hexyl, isopropyl, isobutyl, tertiary butyl, isopentyl, 2-methylbutyl and 3-methylbutyl which are each unsubstituted or substituted by 1, 2 or 3 substituents selected from the group consisting of oxo, —F, —NO₂, —CN, —CF₃, —C(═O)CH₃, —C(═O)OCH₃, —NH—C(═O)(CH₃), —CH₂-phenyl, -phenyl; and cyclopropyl, cyclobutyl, cyclopentyl, cyclohexyl, cyclohexenyl and cyclooctyl which are each unsubstituted or substituted by 1, 2, 3, or 4 substituents selected from the group consisting of methyl, ethyl, 1-propyl, 1-butyl, 1-pentyl, 1-hexyl, isopropyl, isopropenyl, isobutyl, tertiary butyl, isopentyl, 2-methylbutyl, 3-methylbutyl, -methoxy, -ethoxy, —F, —NO₂, —CN, —CF₃, —C(═O)CH₃, —C(═O)OCH₃, —NH—C(═O)CH₃.
3. The process according to embodiment 1, wherein R₂ is hydrogen or selected from group consisting of methyl, ethyl, 1-propyl, 1-butyl, 1-pentyl, 1-hexyl, isopropyl, isobutyl, tertiary butyl, isopentyl, 2-methylbutyl, 3-methylbutyl, cyclopropyl, cyclobutyl, cyclopentyl, cyclohexyl and cyclooctyl which are each unsubstituted.
4. The process according to embodiment 1, wherein R₃ is selected from group consisting of hydrogen, methyl, ethyl, 1-propyl, 1-butyl, 1-pentyl, 1-hexyl, isopropyl, isobutyl, tertiary butyl, isopentyl, 2-methylbutyl and 3-methylbutyl.
5. The process according to embodiment 1, wherein R₄ is selected from group consisting of methyl, ethyl, 1-propyl, 1-butyl, 1-pentyl, 1-hexyl, isopropyl, isobutyl, tertiary butyl, isopentyl, 2-methylbutyl and 3-methylbutyl.
6. The process according to any embodiment 1 or 2, wherein R1 is cyclohexyl or cyclohexenyl which is unsubstituted or substituted by 1, 2 or 3 substituents selected from the group consisting of oxo, methyl, ethyl, 1-propyl, 1-butyl, 1-pentyl, 1-hexyl, isopropyl, isopropenyl, isobutyl, tertiary butyl, isopentyl, 2-methylbutyl and 3-methylbutyl.
7. The process according to embodiment 1 or 2, wherein R₂ is hydrogen or selected from group consisting of methyl, ethyl, 1-propyl, 1-butyl, 1-pentyl, 1-hexyl, isopropyl, isobutyl, tertiary butyl, isopentyl, 2-methylbutyl and 3-methylbutyl which are each unsubstituted.
8. The process according to embodiment 1, wherein the imidazole of formula (III) is selected from the group consisting of 1-methyl imidazole, 1-ethyl imidazole, 1-propyl imidazole, 1-isopropyl imidazole, 1-butyl imidazole, and 1,2-dimethyl imidazole.
9. The process according to embodiment 8, wherein the imidazole of formula (III) is 1,2-dimethyl imidazole or 1-methyl-imidazole.
10. The process according to any one of embodiments 1 to 9, wherein in step a) the molar ratio of the imidazole of formula (III) to the compound of formula (II) is in the range of ≥0.05:1 to ≤3.0:1.
11. The process according to any one of embodiments 1 to 10, wherein in step a) the temperature is in the range of ≥10° C. to ≤80° C.
12. The process according to any one of embodiments 1 to 11, wherein at least one of step a) and step b) is carried out in the presence of at least one non-polar solvent.
13. The process according to embodiment 12, wherein the at least one non-polar solvent has dielectric constant in the range of ≥1.5 to ≤6.0.
14. The process according to embodiment 12 or 13, wherein the at least one non-polar organic solvent is selected from the group consisting of aliphatic hydrocarbon, aromatic hydrocarbon and ether.
15. The process according to embodiment 14, wherein the aliphatic hydrocarbon is selected from the group consisting of pentane, hexane, heptane, cyclohexane and petroleum ether.
16. The process according to embodiment 14, wherein the aromatic hydrocarbon is selected from the group consisting of benzene, toluene and xylene.
17. The process according to embodiment 14, wherein the ether is selected from the group consisting of diethyl ether, diisopropyl ether, diethylene glycol dimethyl ether and methyl tert-butyl ether.
18. The process according to any of embodiments 12 to 17, wherein the at least one non-polar solvent is selected from the group consisting of toluene, xylene, cyclohexane, heptane and methyl tert-butyl ether.
19. The process according to any one of embodiments 1 to 18, wherein in step b) the molar ratio of the compound of formula (II) to the compound of formula (V) is in the range of ≥1:2 to ≤1:20.
20. The process according to any one of embodiments 1 to 19, wherein in step b) the temperature is in the range of ≥20° C. to ≤60° C.
21. The process according to any one of the preceding embodiments, wherein step a) and step b) are carried out simultaneously.
22. The process according to any one of embodiments 12 to 21, wherein the compound of formula (IV) is isolated from the at least one non-polar solvent.
23. The process according to any one of the preceding embodiments, wherein the compound of general formula (II) is obtained by reacting a compound of formula (II′) with phosgene

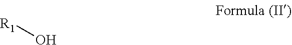

wherein
R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl.
24. The process according to any one of embodiments 1 to 23, for preparing the compound of formula (IA),

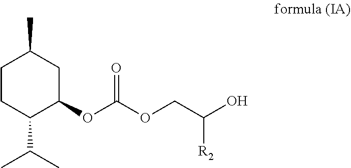

wherein R₂ is hydrogen or methyl;
whereby if R₂ is methyl, the formula (IA) comprises
the compound of formula (Ia)

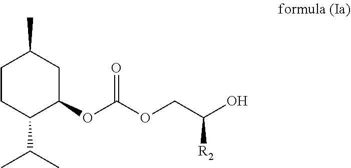

the compound of the formula (Ib)

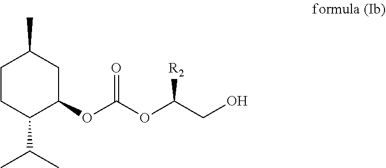

the compound of formula (Ic)

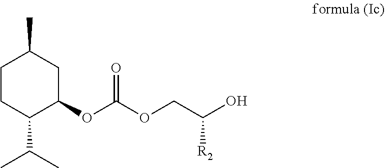

the compound of formula (Id)

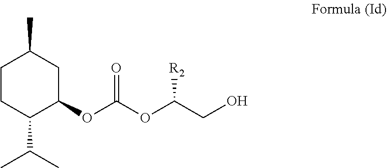

and its stereoisomers,
comprising at least the steps of:
b) reacting the compound of formula (IIA),

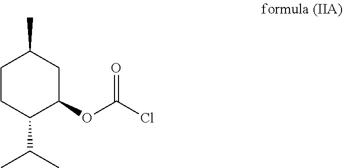

with an imidazole of formula (III)

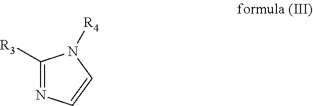

wherein R₃ is hydrogen or unsubstituted, linear or branched, C₁-C₆-alkyl and R₄ is unsubstituted, linear or branched C₁-C₆-alkyl;
to obtain a compound of formula (IVA),

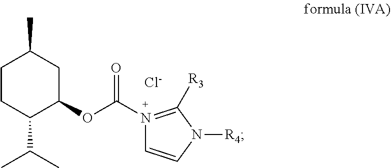

wherein R₃ is hydrogen or unsubstituted, linear or branched, C₁-C₆-alkyl and R₄ is unsubstituted, linear or branched C₁-C₆-alkyl;
and
b) reacting the compound of formula (IVA) with the compound of formula (VA) and its stereoisomers;

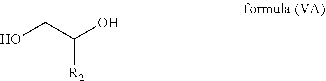

wherein R₂ is hydrogen or methyl;
to obtain the compound of formula (IA) and its stereoisomers.
25. A process according to any one the preceding embodiments, wherein the compound of general formula (IA) is purified by
   subjecting the crude mixture to steam stripping to obtain a stripped mixture; and distillation of the stripped mixture of step a) by short path evaporation to obtain the purified carbonic esters of formula (IA).
26. The process according to embodiment 1 or 24, wherein said compound of formula (I) and formula (IA), respectively, is

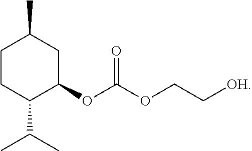 [SMILES: CC(C)[C@@H]1CC[C@@H](C)C[C@H]1OC(=O)OCCO]

27. The process according to embodiment 1 or 24, wherein said compound of formula (I) and formula (IA), respectively, is

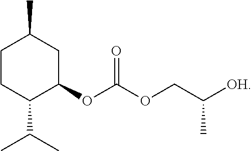 [SMILES: CC(C)[C@@H]1CC[C@@H](C)C[C@H]1OC(=O)OC[C@@H](C)O]

28. The process according to embodiment 1 or 24, wherein said compound of formula (I) and formula (IA), respectively, is

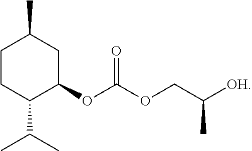 [SMILES: CC(C)[C@@H]1CC[C@@H](C)C[C@H]1OC(=O)OC[C@H](C)O]

29. A compound of formula (IVa) and stereoisomers,

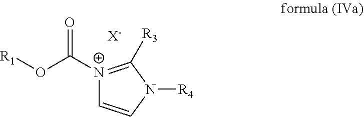

wherein,
when R₃ is unsubstituted, linear or branched C₁-C₆-alkyl; R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; or
wherein when R₃ is hydrogen; R₁ is selected from the group consisting of unsubstituted or substituted, linear or branched C₄-C₁₀-alkyl or -alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl; and
Rₑ is unsubstituted, linear or branched C₁-C₁₀-alkyl;
and X is chloride or bromide.
30. The compound according to embodiment 28, wherein when R₃ is methyl; R₁ is selected from the group consisting of unsubstituted, linear C₁-C₁₀-alkyl, unsubstituted or substituted C₅-C₁₀-cycloalkyl and unsubstituted or substituted C₅-C₁₀-cycloalkenyl; or
   wherein when R3 is hydrogen; R1 is linear or branched C4-C10-alkyl or unsubstituted or substituted C5-C10-cycloalkyl or unsubstituted or substituted C5-C10-cycloalkenyl.
31. The compound according to embodiment 28 or 29, wherein the compound of formula (IVa) is

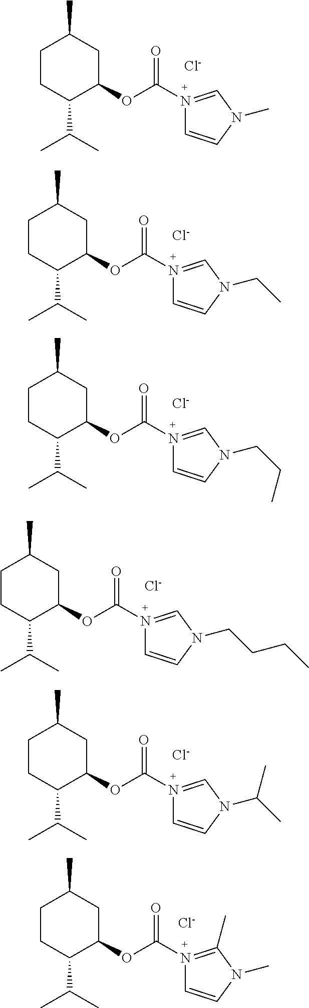

32. Use of the compound of formula (IV),

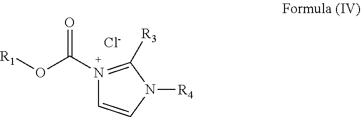

wherein
R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;
R₃ is hydrogen or unsubstituted, linear or branched, C₁-C₆-alkyl; and
R₄ is unsubstituted, linear or branched C₁-C₆-alkyl;
to prepare the compound of formula (I)

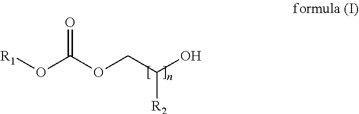

wherein
R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; R₂ is selected from the group consisting of hydrogen, unsubstituted or substituted, linear or branched C₁-C₁₀-alkyl, unsubstituted or substituted, linear or branched C₂-C₁₀alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;
n is 1, 2 or 3; and
wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl.
33. A method of preparing the compound of formula (I) as defined in embodiment 32 comprising using the compound of formula (IV) as defined in embodiment 32.

### EXAMPLES

The characterization is done by ¹H-NMR or by ¹³C-NMR measurements on a Bruker DPX 500 spectrometer and by High Resolution Mass Spectroscopy on a Thermo Fischer QExactive plus machine. ¹H-NMR and ¹³C-NMR: The signals are characterized by chemical shift (ppm) vs. tetramethylsilane, by their multiplicity and by their integral (relative number of hydrogen atoms given). The following abbreviations are used to characterize the multiplicity of the signals: m=multiplet, q=quartet, t=triplet, d=doublet and s=singlet. Abbreviations used are: h for hour(s), min for minute(s), rt for retention time and ambient temperature for 20-25° C.

### General Procedures

### Example 1—Preparation of chloride salt of (1R,2S,5R)-2-isopropyl-5-methyl-cyclohexyl 3-methylimidazol-1-ium-1-carboxylate (table 1, entry 1)

Heptane (600 mL) and 1-methylimidazole (0.229 Mol) were placed in a 1 L reactor at 25° C. The respective chloroformate (0.218 Mol) was added within 2.0 hours and stirred for 1 hour after complete addition. The obtained suspension was filtered and the solid was washed with additional heptane (150 mL). The resulting white, hygroscopic salt was carefully dried at 30° C.

Examples 2-7: The following examples in Table 1 further illustrate the process for the preparation of compound of formula (IV) of the present invention and do not restrict the invention in any manner.

TABLE 1

| | Compound of | | | |
|---|---|---|---|---|
| S.No. | formula (IV) | Solvent | Yield | Characterization data |
| 1. | 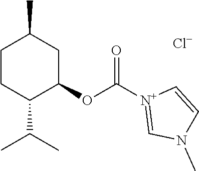 [SMILES: CC(C)[C@@H]1CC[C@@H](C)C[C@H]1OC(=O)[n+]1ccn(C)c1 ; [Cl-]] | Heptane | 91% | ¹H-NMR (500 MHz, d₆-DMSO): δ = 10.0- 9.99 (m, 1H), 8.14 (t, J = 2.0 Hz, 1H), 7.92 (t, J = 1.9 Hz, 1H), 4.89 (td, J = 10.9, 4.5 Hz, 1H), 3.95 (s, 3H), 2.16- 2.11 (m, 1H), 1.99-1.91 (m, 1H), 1.73- 1.65 (m, 2H), 1.60-1.53 (m, 2H), 1.19 (q, J = 12.0 Hz, 1H), 1.11 (qd, J = 13.0, 3.3 Hz, 1H), 0.92 (d, J = 6.5 Hz, 3H), 0.89 (d, J = 7.0 Hz, 3H), 0.88-0.84 (m, 1H), 0.76 (d, J = 6.9 Hz, 3H) ppm. ¹³C-NMR (125 MHz, d₆-DMSO): δ = |
| | | | | 145.4, 138.6, 124.5, 119.7, 81.3, 46.2, |
| | | | | 39.6, 36.4, 33.2, 30.6, 25.3, 22.4, 21.7, |
| | | | | 20.5, 16.0 ppm. |
| | | | | HRMS (ESI⁺): calc′d for C₁₅H₂₅N₂O₂ |
| | | | | 265.1911 [M]⁺, found 265.1913. |
| 2. | 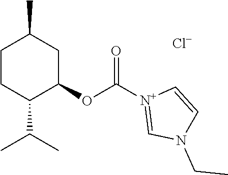 [SMILES: CCn1cc[n+](C(=O)O[C@@H]2C[C@H](C)CC[C@H]2C(C)C)c1 ; [Cl-]] | Methyl tert- butyl ether | 97% | ¹H-NMR (500 MHz, d₆-DMSO): δ = 10.40- 10.38 (m, 1H), 8.27 (dd, J = 2.3, 1.5 Hz, 1H), 8.19 (t, J = 2.0 Hz, 1H), 4.86 (td, J = 10.9, 4.5 Hz, 1H), 4.46-4.36 (m, 2H), 2.14-2.08 (m, 1H), 2.00-1.91 (m, 1H), 1.70-1.63 (m, 2H), 1.60-1.49 (m, 2H), 1.44 (t, J = 7.3 Hz, 3H), 1.19 (q, J = 11.9 Hz, 1H), 1.09 (qd, J = 13.2, 3.6 Hz, 1H), 0.89 (d, J = 6.6 Hz, 3H), 0.87 (d, J = 7.0 Hz, 3H), 0.86-0.83 (m, 1H), 0.74 (d, J = 7.0 Hz, 3H) ppm. |
| | | | | ¹³C-NMR (125 MHz, d₆-DMSO): δ = |
| | | | | 145.5, 138.2, 123.3, 120.0, 81.1, 46.1, |
| | | | | 44.9, 39.7, 33.3, 30.6, 25.3, 22.5, 21.7, |
| | | | | 20.6, 16.0, 15.0 ppm. |
| | | | | HRMS (ESI⁺): calc′d for C₁₆H₂₇N₂O₂ |
| | | | | 279.2067 [M]⁺, found 279.2071. |
| 3. | 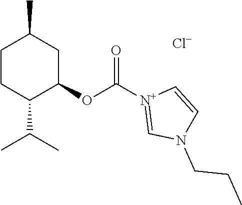 [SMILES: CCCn1cc[n+](C(=O)O[C@@H]2C[C@H](C)CC[C@H]2C(C)C)c1 ; [Cl-]] | Methyl tert- butyl ether | 80% | ¹H-NMR (500 MHz, d₆-DMSO): δ = 10.18 (t, J = 1.5 Hz, 1H), 8.19-8.17 (m, 1H), 8.12-8.10 (m, 1H), 4.89 (td, J = 10.9, 4.5 Hz, 1H), 4.29 (t, J = 7.1 Hz, 2H), 2.16-2.11 (m, 1H), 2.00-1.94 (m, 1H), 1.90-1.82 (m, 2H), 1.71-1.66 (m, 2H), 1.60-1.52 (m, 2H), 1.20 (q, J = 11.9 Hz, 1H), 1.11 (qd, J = 13.6, 3.6 Hz, 1H), 0.92- 0.86 (m, 10H), 0.76 (d, J = 6.9 Hz, 3H) ppm. ¹³C-NMR (125 MHz, d6-DMSO): δ = 145.5, 138.2, 123.3, 120.1, 81.2, 50.8, 46.1, 39.6, 33.3, 30.6, 25.3, 22.5, 22.4, |
| | | | | 21.7, 20.5, 16.0, 10.3 ppm. |
| | | | | HRMS (ESI⁺): calc′d for C₁₇H₂₉N₂O₂ |
| | | | | 293.2224 [M]⁺, found 293.2228. |
| 4. | 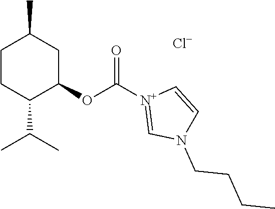 [SMILES: CCCCn1cc[n+](C(=O)O[C@@H]2C[C@H](C)CC[C@H]2C(C)C)c1 ; [Cl-]] | Methyl tert- butyl ether | 96% | ¹H-NMR (500 MHz, d₆-DMSO): δ = 10.25 (t, J = 1.6 Hz, 1H), 8.18 (t, J = 2.0 Hz, 1H), 8.14 (dd, J = 2.3 Hz, 1H), 4.88 (td, J = 10.9, 4.5 Hz, 1H), 4.34 (t, J = 7.2 Hz, 2H), 2.14-2.12 (m, 1H), 2.01-1.94 (m, 1H), 1.85-1.79 (m, 2H), 1.71-1.66 (m, 2H), 1.60-1.51 (m, 2H), 1.32-1.24 (m, 2H), 1.23-1.16 (m, 1H), 1.10 (qd, J = 13.5, 3.5 Hz, 1H), 0.91-0.88 (m, 10H), 0.76 (d, J = 6.9 Hz, 3H) ppm. ¹³C-NMR (125 MHz, d₆-DMSO): δ = 145.5, 138.3, 123.4, 120.1, 81.2, 49.2, 46.1, 38.9, 33.3, 31.0, 30.6, 25.3, 22.5, |
| | | | | 21.7, 20.5, 18.6, 16.0, 13.3 ppm. |
| | | | | HRMS (ESI⁺): calc′d for C₁₈H₃₁N₂O₂ |
| | | | | 307.2380 [M]⁺, found 307.2384. |
| 5. | 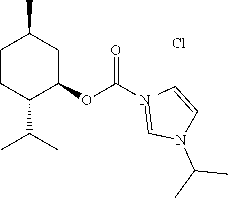 [SMILES: CC(C)[C@@H]1CC[C@@H](C)C[C@H]1OC(=O)[n+]1ccn(C(C)C)c1 ; [Cl-]] | Methyl tert- butyl ether | 95% | ¹H-NMR (500 MHz, d₆-DMSO): δ = 10.15- 10.13 (m, 1H), 8.26 (t, J = 1.9 Hz, 1H), 8.21 (t, J = 2.0 Hz, 1H), 4.92-4.84 (m, 2H), 2.14-2.09 (m, 1H), 2.01-1.93 (m, 1H), 1.71-1.66 (m, 2H), 1.61-1.54 (m, 2H), 1.51 (d, J = 6.7 Hz, 3H), 1.50 (d, J = 6.7 Hz, 3H), 1.21 (q, J = 11.8 Hz, 1H), 1.11 (qd, J = 13.2, 3.7 Hz, 1H), 0.91 (d, J = 6.6 Hz, 3H), 0.89 (d, J = 7.0 Hz, 3H), 0.88-0.84 (m, 1H), 0.76 (d, J = 6.9 Hz, 3H) ppm. ¹³C-NMR (125 MHz, d₆-DMSO): δ = 145.5, 137.2, 121.2, 120.3, 81.1, 53.3, |
| | | | | 46.1, 39.6, 33.3, 30.6, 25.3, 22.4, 21.8, |
| | | | | 21.74, 21.68, 20.5, 15.9 ppm. |
| | | | | HRMS (ESI⁺): calc′d for C₁₇H₂₉N₂O₂ |
| | | | | 293.2224 [M]⁺, found 293.2226. |
| 6. | 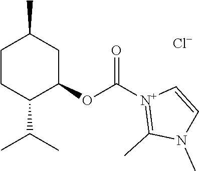 [SMILES: Cc1n(C)cc[n+]1C(=O)O[C@@H]1C[C@H](C)CC[C@H]1C(C)C ; [Cl-]] | Methyl tert- butyl ether | 97% | ¹H-NMR (500 MHz, d₆-DMSO): δ = 8.03 (d, J = 2.4 Hz, 1H), 7.80 (d, J = 2.4 Hz, 1H), 4.89 (td, J = 11.0, 4.5 Hz, 1H), 3.84 (s, 3H), 2.80 (s, 3H), 2.15-2.10 (m, 1H), 1.94-1.88 (m, 1H), 1.73-1.65 (m, 2 H), 1.63-1.51 (m, 2H), 1.22 (q, J = 12.0 Hz, 1H), 1.12 (qd, J = 13.4, 3.7 Hz, 1H), 0.92 (d, J = 6.5 Hz, 3H), 0.90 (d, J = 7.0 Hz, 3H), 0.88-0.84 (m, 1H), 0.77 (d, J = 6.9 Hz, 3H) ppm. ¹³C-NMR (125 MHz, d₆-DMSO): δ = |
| | | | | 148.9, 146.3, 122.8, 119.2, 81.0, 46.0, |
| | | | | 39.4, 35.1, 33.2, 30.6, 25.6, 22.5, 21.7, |
| | | | | 20.5, 16.1, 12.5 ppm. |
| | | | | HRMS (ESI⁺): calc′d for C₁₆H₂₇N₂O₂ |
| | | | | 279.2067 [M]⁺, found 279.2070. |
| 7. | 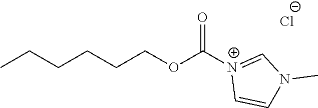 | Methyl tert- butyl ether | 84% | ¹H-NMR (500 MHz, d₆-DMSO): δ = 9.94- 9.92 (m, 1H), 8.14 (t, J = 2.0 Hz, 1H), 7.89 (t, J = 1.9 Hz, 1H), 4.49 (t, J = 6.5 Hz, 2H), 3.94 (s, 3H), 1.78-1.72 (m, 2H), 1.34-1.24 (m, 6H), 0.90-0.84 (m, 3H) ppm. ¹³C-NMR (125 MHz, d₆-DMSO): δ = 145.9, |
| | | | | 138.6, 124.6, 119.9, 70.4, 36.5, 30.8, |
| | | | | 27.7, 24.7, 21.9, 13.9 ppm. |

### Example 9—Preparation of menthylpropyleneglycolcarbonate

In a first 1 L double-jacketed reactor with overhead stirrer, toluene (600 mL) and 1,2-dimethylimidazole (33 g, 0.34 Mol) were placed at 25° C. Menthylchloroformate (75.1 g, purity approx. 96%, approx. 0.33 Mol) which contained 2.5 w % menthylchloride (corresponding to 1.88 g) was added over 2 h at 25° C. After complete addition, stirring was continued for 30 min. The acyl-imidazolium-salt precipitated and the resulting suspension was filtered and the solid was washed twice with toluene (3×300 mL). The mother liquor and the toluene of the two washing steps contained the menthylchloride (2.02 g). The acyl-imidazolium salt, essentially free of menthylchloride, was resuspended in toluene (300 ml). In a second 1 L double-jacketed reactor, 1,2-propanediol (248.8 g, 3.27 Mol) and 1,2-dimethylimidazole (1.1 g, 0.01 Mol) were placed at 50° C. The suspension from the first reactor was then dosed into the second reactor over 90 min at 50° C. After complete addition, stirring was continued at 50° C. for 30 min.

Then, the biphasic reaction mixture was cooled to 25° C. and the phases were separated. The glycol-phase was reextracted twice with toluene (2×60 mL) and the united toluene phases were washed with 5% aq. NaHCO₃-solution (300 mL) and water (2×300 mL). The solvent was removed using a thin-film evaporator (70° C., 180 mbar) and the product was obtained as a clear viscous liquid (76% yield for the mixture of products 1 and 2 in table 2). The remaining menthylchloride content was 0.01%.

### Example 10—Purification of Menthylpropyleneglycolcarbonate (MPC)

The crude menthylpropyleneglycolcarbonate (having the following components, Toluene −56.52 wt. %, Menthol-1.06 wt. %, menthyl chloride-0.21 wt. %, MPC-40.37 wt. %, dimer 1.46 wt %) was subjected to steam stripping in a column where the sump temperature does not exceed 120° C. and the pressure was between 0.1 and 0.2 bar. Following steam stripping Menthylpropyleneglycolcarbonate and other impurities were separated as bottom product and toluene and menthyl chloride as the head product.

The head product was subjected to phase separation to separate water phase and toluene phase. The toluene phase was further distilled using a distillation column at a sump temperature of 71° C. and head temperature of 43° C. and a pressure of 0.1 bar to separate menthyl chloride and menthol.

Menthylpropyleneglycolcarbonate was distillated overhead to separate dimer and impurities using a short path evaporator. The pressure was in the range of 0.16-0.4 bar and temperature between 119-124° C. The total yield of Menthylpropyleneglycolcarbonate in laboratory experiments was between 85-91%.

With appropriate modification of the starting materials or intermediates, the procedures as described in the example above was used to obtain further compounds of formula (I). The compounds obtained in this manner are listed in the Table 2 that follows, together with physical data.

TABLE 2

| S.No. | Compound of formula (I) | Characterization data |
|---|---|---|
| 1. | 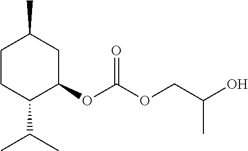 [SMILES: CC(O)COC(=O)O[C@@H]1C[C@H](C)CC[C@H]1C(C)C] | ¹³C-NMR (125 MHz, CDCl₃): δ = 154.9, 78.7, 72.5, 65.9, 47.0, 40.7, 34.0, 31.4, 26.0, 23.2, 22.0, 20.7, 18.8, 16.2 ppm. |
| 2. | 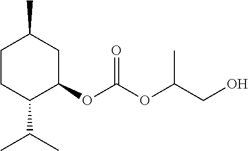 [SMILES: CC(CO)OC(=O)O[C@@H]1C[C@H](C)CC[C@H]1C(C)C] | ¹³C-NMR (125 MHz, CDCl₃): δ = 154.1, 77.0, 75.1, 63.5, 46.6, 40.5, 33.7, 30.9, 26.0, 23.1, 21.9, 20.4, 16.3, 16.2 ppm. |
| 3. | 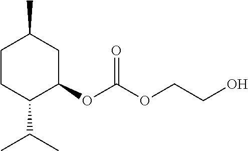 [SMILES: CC(C)[C@@H]1CC[C@@H](C)C[C@H]1OC(=O)OCCO] | ¹H-NMR (500 MHz, CDCl₃): δ = 4.52 (td, J = 10.9, 4.5 Hz, 1H), 4.29-4.21 (m, 2H), 3.86-3.83 (m, 2H), 2.10-2.05 (m, 2H), 2.00-1.91 (m, 1H), 1.70-1.65 (m, 2H), 1.53-1.44 (m, 1H), 1.43- 1.37 (m, 1H), 1.09-1.01 (m, 2H), 0.91 (d, J = 6.6 Hz, 3H), 0.90 (d, J = 7.0 Hz, 3H), 0.88-0.82 (m, 1H), 0.78 (d, J = 7.0 Hz, 3H) ppm. ¹³C-NMR (125 MHz, CDCl₃): δ = 155.0, 78.7, 69.1, 61.1, 47.0, 40.7, 34.0, 31.4, 26.0, 23.2, 22.0, 20.7, 16.2 ppm. |

### Example 11: Preparation of menthyl-ethylene glycol-carbonate

Ethylene glycol (540 g, 8.70 mol, 10 eq.) and 1-methylimidazole (157 g, 1.91 mol, 2.2 eq.) were placed in a reactor at 25° C. and a solution of menthylchloroformate (191 g, 0.87 mol, 1.0 eq.) in toluene (99 g) was added over 2 hours. Stirring was continued for 2 h after complete addition. Then, stirring was stopped and the two phases of the reaction mixture were separated. The lower phase (glycol phase, 690 g) was washed with toluene (2×120 g). The toluene phases and the upper phase of the reaction mixture were combined (535 g) and washed with aqueous HCl (200 g water containing 30 g HCl 32%) followed by aqueous NaHCO₃ solution (200 mL). After removal of the solvent, menthyl-ethylene glycol-carbonate was obtained in 84% yield.

Recovery of excess ethylene glycol and 1-methylimidazole:aqueous sodium hydroxide solution (25%, 139 g) was added to the lower phase of the step above (650 g; after extraction with toluene) to adjust the pH of the mixture to 10-12. The mixture was distilled on an agitated thin-film evaporator (75° C., 100 mBar) to remove water and small amounts of toluene. The sump was diluted with PEG 400 (60 g) and distilled again on an agitated thin-film evaporator (130° C., 1 mBar). The distillate contained ethylene glycol and 1-methyl-imidazole.

### Example 12: Preparation of menthyl-ethylene glycol-carbonate

Example 11 was repeated using 1,2-dimethylimidazole as the base.

### Comparative Example

A. Attempted Preparation of acyl-pyridinium Salt

Heptane (600 mL) and pyridine (0.220 Mol) were placed in a 1 L reactor at 25° C. Methylchloroformate (0.218 Mol) was added slowly over 2.0 hours. After complete addition, stirring was continued at 25° C. for 1.0 hour. A small amount of precipitate was formed, which was filtered off, washed with heptane, and dried at 30° C. on a rotary evaporator. The material turned out to be a mixture of menthol and pyridine hydrochloride. 13C-NMR did not show a carbonyl signal.

1.-16. (canceled)

17. A process for preparing a carbonic acid ester of formula (I) and its stereoisomers, 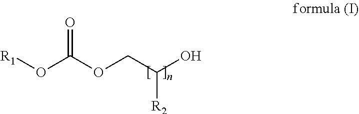
wherein
R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl and unsubstituted or substituted C₅-C₁₀-cycloalkyl and unsubstituted or substituted C₅-C₁₀-cycloalkenyl;
R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀cycloalkyl and unsubstituted or substituted C₅-C₁₀-cycloalkenyl;
n is 1, 2 or 3;
wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;
comprising at least the steps of:
a) reacting a compound of formula (II) 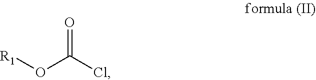
wherein
R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl with an imidazole of formula (III), 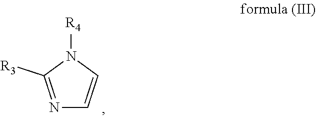
wherein
R₃ is hydrogen or unsubstituted, linear or branched, C₁-C₆-alkyl and R₄ is unsubstituted, linear or branched, C₁-C₆-alkyl;
to obtain a compound of formula (IV), 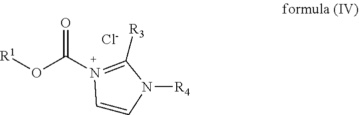
wherein
R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;
R₃ is hydrogen or unsubstituted, linear or branched, C₁-C₆-alkyl; and
R₄ is unsubstituted, linear or branched, C₁-C₆-alkyl;
and
b) reacting the compound of formula (IV) with a compound of formula (V) 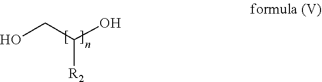
wherein
R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀cycloalkyl and unsubstituted or substituted C₅-C₁₀-cycloalkenyl;
n is 1, 2 or 3; and
wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl;
to obtain a compound of formula (I) and its stereoisomers.

18. The process according to claim 17, wherein R₁ is selected from group consisting of methyl, ethyl, 1-propyl, 1-butyl, 1-pentyl, 1-hexyl, isopropyl, isobutyl, tertiary butyl, isopentyl, 2-methylbutyl and 3-methylbutyl which are each unsubstituted or substituted by 1, 2 or 3 substituents selected from the group consisting of oxo, —F, —NO₂, —CN, —CF₃, —C(═O)CH₃, —C(═O)OCH₃, —NH—C(═O)(CH₃), —CH₂-phenyl, -phenyl; and
cyclopropyl, cyclobutyl, cyclopentyl, cyclohexyl, cyclohexenyl and cyclooctyl which are each unsubstituted or substituted by 1, 2, 3, or 4 substituents selected from the group consisting of methyl, ethyl, 1-propyl, 1-butyl, 1-pentyl, 1-hexyl, isopropyl, isopropenyl, isobutyl, tertiary butyl, isopentyl, 2-methylbutyl, 3-methylbutyl, -methoxy, -ethoxy, —F, —NO₂, —CN, —CF₃, —C(═O)CH₃, —C(═O)OCH₃, —NH—C(═O)CH₃.

19. The process according to claim 17, wherein R₂ is hydrogen or selected from the group consisting of methyl, ethyl, 1-propyl, 1-butyl, 1-pentyl, 1-hexyl, isopropyl, isobutyl, tertiary butyl, isopentyl, 2-methylbutyl, 3-methylbutyl, cyclopropyl, cyclobutyl, cyclopentyl, cyclohexyl and cyclooctyl which are each unsubstituted.

20. The process according to claim 17, wherein R₃ is selected from group consisting of hydrogen, methyl, ethyl, 1-propyl, 1-butyl, 1-pentyl, 1-hexyl, isopropyl, isobutyl, tertiary butyl, isopentyl, 2-methylbutyl and 3-methylbutyl.

21. The process according to claim 17, wherein R₄ is selected from group consisting of methyl, ethyl, 1-propyl, 1-butyl, 1-pentyl, 1-hexyl, isopropyl, isobutyl, tertiary butyl, isopentyl, 2-methylbutyl and 3-methylbutyl.

22. The process according to claim 17, wherein the imidazole of formula (III) is selected from the group consisting of 1-methyl imidazole, 1-ethyl imidazole, 1-propyl imidazole, 1-isopropyl imidazole, 1-butyl imidazole, and 1,2-dimethyl imidazole.

23. The process according to claim 17, wherein at least one of step a) and step b) is carried out in the presence of at least one non-polar solvent.

24. The process according to claim 23, wherein the at least one non-polar organic solvent is selected from the group consisting of aliphatic hydrocarbon, aromatic hydrocarbon and ether.

25. The process according to claim 17, wherein step a) and step b) are carried out simultaneously.

26. The process according to claim 23, wherein the compound of formula (IV) is isolated from the at least one non-polar solvent.

27. The process according to claim 17, wherein the compound of general formula (II) is obtained by reacting a compound of formula (II′) with phosgene 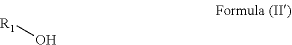
wherein
R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl.

28. The process according to claim 17, for preparing the compound of formula (IA), 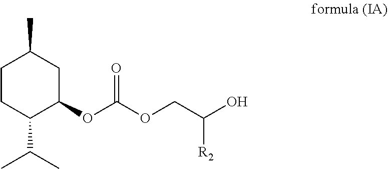
wherein R₂ is hydrogen or methyl;
whereby if R₂ is methyl, the formula (IA) comprises
the compound of formula (Ia) 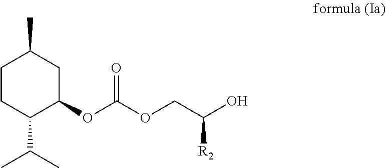
the compound of the formula (Ib) 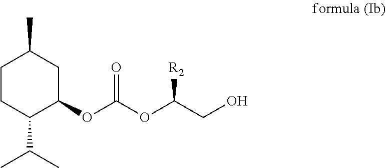
the compound of formula (Ic) 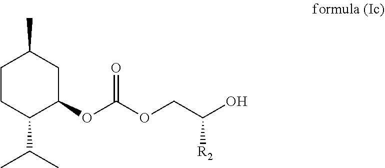
the compound of formula (Id) 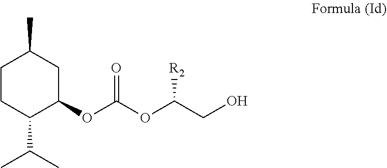
and its stereoisomers,
comprising at least the steps of:
reacting the compound of formula (IIA), 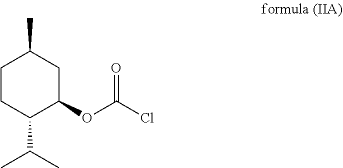
with an imidazole of formula (III) 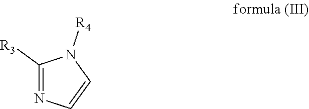
wherein R₃ is hydrogen or unsubstituted, linear or branched, C₁-C₆-alkyl and R₄ is unsubstituted, linear or branched C₁-C₆-alkyl;
to obtain a compound of formula (IVA), 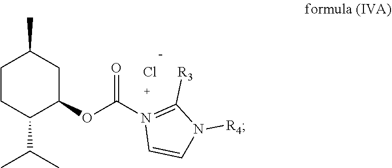
wherein R₃ is hydrogen or unsubstituted, linear or branched, C₁-C₆-alkyl and R₄ is unsubstituted, linear or branched C₁-C₆-alkyl;
and
b) reacting the compound of formula (IVA) with the compound of formula (VA) and its stereoisomers; 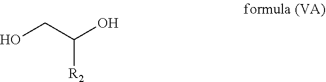
wherein R₂ is hydrogen or methyl;
to obtain the compound of formula (IA) and its stereoisomers.

29. A process according to claim 17, wherein the compound of general formula (IA) is purified by;
(a) subjecting the crude mixture to steam stripping to obtain a stripped mixture; and
(b) distillation of the stripped mixture of step a) by short path evaporation to obtain the purified carbonic esters of formula (IA).

30. A compound of formula (IVa) and stereoisomers, 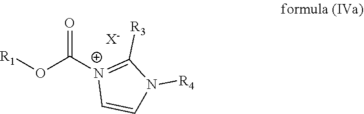
wherein,
when R₃ is unsubstituted, linear or branched C₁-C₆-alkyl; R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl; or
wherein when R₃ is hydrogen; R₁ is selected from the group consisting of unsubstituted or substituted, linear or branched C₄-C₁₀-alkyl or -alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl; and
R₄ is unsubstituted, linear or branched C₁-C₁₀-alkyl;
and X is chloride or bromide.

31. The compound according to claim 30, wherein the compound of formula (IVa) is

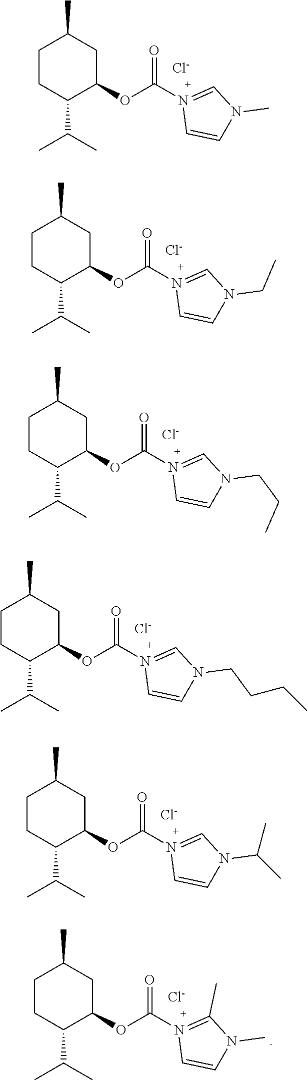

32. Use of the compound of formula (IV), 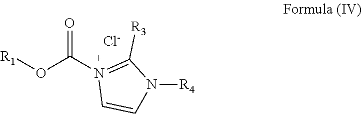
wherein
R¹ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;
R₃ is hydrogen or unsubstituted, linear or branched, C₁-C₆-alkyl; and
R₄ is unsubstituted, linear or branched C₁-C₆-alkyl;
to prepare the compound of formula (I) 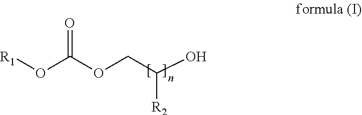
wherein
R₁ is selected from the group consisting of unsubstituted or substituted, linear or branched C₁-C₁₀-alkyl, unsubstituted or substituted, linear or branched C₃-C₁₀-alkenyl, unsubstituted or substituted, linear or branched C₃-C₁₀-alkynyl, unsubstituted or substituted C₅-C₁₀-cycloalkyl and unsubstituted or substituted C₅-C₁₀-cycloalkenyl;
R₂ is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀cycloalkyl unsubstituted or substituted C₅-C₁₀-cycloalkenyl;
n is 1, 2 or 3; and
wherein when n is 2 or 3; R₂, independently, is selected from the group consisting of hydrogen, unsubstituted or substituted, linear or branched C₁-C₁₀-alkyl, unsubstituted or substituted, linear or branched C₂-C₁₀-alkenyl, unsubstituted or substituted, linear or branched C₂-C₁₀-alkynyl, unsubstituted or substituted C₅-C₁₀-cycloalkyl and unsubstituted or substituted C₅-C₁₀-cycloalkenyl.
